(19) United States
(12) Patent Application Publication
(10) Pub. No.: US 2023/0085194 A1
(43) Pub. Date: Mar. 16, 2023

(54) Adsorptive Radiocontrast Constructs And Uses Thereof

(71) Applicant: Eslam Abbas Baseuny, Cairo (EG)

(72) Inventor: Eslam Abbas Baseuny, Cairo (EG)

(21) Appl. No.: 17/944,142

(22) Filed: Sep. 13, 2022

Related U.S. Application Data
(60) Provisional application No. 63/243,272, filed on Sep. 13, 2021.

(51) Int. Cl. A61K 45/06; A61K 38/00; A61K 9/00; A61K 9/50; B82Y 40/00

(52) U.S. Cl. CPC A61K 45/06; A61K 38/00; A61K 9/0024; A61K 9/5094; B82Y 40/00; A61K 2121/00

(57) ABSTRACT

The presently-disclosed subject matter relates to adsorptive radiocontrast constructs in multiple advantageous structural embodiments, which are designed to possess dual therapeutic and diagnostic actions. A concomitant therapeutic antiplatelet, as well as vasodilatation, effects, and a diagnostic real-time continuous visualization of an artery, can be obtained via direct injection of the constructs into said artery using a suitable catheter during procedural angiography with or without percutaneous intervention.

### CROSS-REFERENCE TO RELATED APPLICATIONS

This application claims the benefit of Provisional Patent Application No. 63/243,272 filed on Sep. 13, 2021 by the present inventor.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### DESCRIPTION

### Field of Invention

The presently-disclosed subject matter relates to adsorptive radiocontrast constructs with dual therapeutic and diagnostic actions that are used for continuous real-time visualization of an artery, wherein said adsorptive radiocontrast constructs are directly injected into said artery via a suitable catheter. The adsorptive radiocontrast constructs have advantageous diagnostic properties as well as therapeutic effects. The presently disclosed subject matter also relates to methods of preparation of said adsorptive radiocontrast constructs, and methods of using the constructs to help a recipient in need thereof.

### Background of the Invention

In medical imaging, radiocontrast agents are substances used to increase the contrast of structures or fluids within the body, as they absorb or alter external electromagnetic radiation, wherein a suitable amount of these agents are usually injected into a vein so that the low-pressure venous system and further dye distribution allow a sufficient time for target tissue visualization using X-ray-based imaging techniques such as computed tomography (contrast CT) and projectional radiography. This is different from radiopharmaceuticals/radiotracers used in nuclear medicine (such as PET and SPECT scans) which emit radiation, and MRI contrast agents which alter the magnetic properties of nearby hydrogen nuclei.

In procedural real-time arterial angiography, with or without percutaneous intervention; a guiding catheter allows for radiopaque (usually iodine-based) dyes to be injected into an artery/arterial tree, so that the location and disease state can be readily assessed using X-ray visualization (such as a digital fluoroscope). During this procedural study/intervention, the injected dye is usually washed away rapidly by the bloodstream of the high-pressure arterial system rendering continuous real-time visualization of the target artery/arterial tree impossible which leads to multiple problems: (i) the interventional procedure becomes more operator-dependent; wherein the interventionist usually estimates the movement of the radiopaque coronary guidewire in relation to the radiolucent arterial wall, so multiple injections of the dye are usually needed to help manipulation of the tip of the guidewire. (ii) Excessive manipulation of the guidewire can lead to several forms of dangerous arrhythmias. (iii) Dissection or perforation of the coronary artery, leading to hemopericardium, can result from faulty manipulation of the guidewire. (iv) Multiple dye injections increase the probability of contrast-induced nephropathy and thyroid dysfunction. (v) Rupture of the atheromatous plaque can result during the manipulation of the guiding catheter, in the aorta, or the guidewire, in the coronaries, leading to endovascular occlusion and consequential infarction.

Therefore, a radiocontrast agent with the ability to adsorb onto the arterial wall for a longer duration can effectively improve real-time visualization of the artery/arterial tree during the intervention. Although the majority of the dye will be washed away rapidly as usual under the high arterial pressure, the arterial wall will be faintly lined with such adsorptive radiocontrast molecules. The interventionist will have the capacity for better manipulation of the densely radiopaque guidewire in relation to the dyed arterial wall, so multiple injections of the dye are not warranted to help the manipulation of the tip of the guidewire.

### SUMMARY OF THE INVENTION

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Those features can be applied to other embodiments of the presently disclosed subject matter, whether listed in this summary or not. To avoid excessive repetition, this summary does not list or suggest all possible combinations of features.

The present invention describes adsorptive radiocontrast constructs, hereinafter abbreviated to ARC(s), wherein several embodiments of said ARCs are described and can be used to help a recipient in need thereof. The proposed ARCs possess dual therapeutic and diagnostic actions, whereby a concomitant therapeutic antiplatelet effect and a diagnostic real-time continuous visualization of an artery can be obtained via direct injection of the proposed ARCs into said artery using a suitable catheter. The structure of the proposed ARCs can comprise, on the scale of small molecules, a radiodense nucleus and one or more adhesive arms tethered to said radiodense nucleus with or without a linker. The therapeutic action of the proposed ARC can be further escalated by the incorporation of one or more additional therapeutically active molecules into its molecular structure, so that said additional therapeutically active molecules can be tethered to the radiodense nucleus, or anchored to or replace the linker.

The linker, also named a spacer, is a flexible molecule or a stretch of molecule of different lengths or polarity that can possess a branching capability, and can be used to link two or more molecules of interest together. While the radiodense nucleus comprises an element or molecule that is capable of absorbing or altering external electromagnetic radiation (radiodensity) resulting in decreased exposure on an electromagnetic waves detector. Whereas, the adhesive arms comprise peptide motifs or peptidomimetics that bind to their corresponding receptors of cell-adhesion molecules, preferentially to integrins. Said peptide motifs are sufficient for cell membrane binding, wherein a minimum amino-acid sequence of said peptide motifs retains the property of cell adhesion. A paramount example of these cell-adhesive peptide motifs, which are both sufficient and indispensable for cell membrane binding (Li et al. 2003), is the tripeptide RGD and its RGD-peptidomimetics (both natural and synthetic) such as RYD (Alon, Bayer, and Wilchek 1990) and KGD (McLane et al. 2004), the GFOGER amino acid sequence (Knight et al. 2000), the YGISR sequence (Boateng et al. 2005), and/or the A5G81 sequence (Zhu et al. 2018), that bind to integrins, which are cell membrane-spanning protein receptors (Van Agthoven et al. 2014).

The radiodense nucleus can be tethered to one adhesive arm resulting in a monovalent ARC or can be tethered to multiple adhesive arms resulting in a multivalent ARC. Additionally, the adhesive arms can be identical resulting in a monospecific ARC, or different resulting in a polyspecific ARC. Accordingly, the combination of both the therapeutic and the diagnostic actions of the proposed ARCs is advantageous and can be used for procedural real-time angiography with or without percutaneous intervention.

Otherwise, the adsorptive radiocontrast construct can comprise one or more adhesive peptides or peptidomimetics that contain within its molecular structure a radiodense element or molecule to achieve the previously-mentioned dual therapeutic and diagnostic actions, wherein the adhesive peptides or peptidomimetics bind to their corresponding receptors of cell-adhesion molecules, preferentially to integrins. These adhesive peptides or peptidomimetics comprise cell-adhesive domains that are sufficient for cell membrane binding, wherein a minimum amino-acid sequence of said cell-adhesive domains retains the property of cell adhesion. Whereas, the radiodense element or molecule is capable of absorbing or altering external electromagnetic radiation resulting in decreased exposure on an electromagnetic waves detector.

Said adhesive peptides or peptidomimetics, that contain within its molecular structure a radiodense element or molecule, can be singles resulting in a monovalent ARC, or can be multiple tethered to each other with flexible linkers resulting in a multivalent ARC. As well, said adhesive peptides or peptidomimetics, that contain within their molecular structure a radiodense element or molecule, can be identical resulting in a monospecific ARC, or different resulting in a polyspecific ARC.

Whereas on the nanoscale, a major embodiment of the present invention is a multivalent adsorptive radiocontrast nanoconstruct, hereinafter abbreviated to MARC(s), that comprises a radiodense nanocore, that is coated with a coat of pluralities of adhesive peptides or peptidomimetics with or without a subcoat of linkers, wherein said pluralities of adhesive peptides or peptidomimetics are presented to interact simultaneously with multiple corresponding binding sites.

The radiodense nanocore comprises an element or molecule that is capable of absorbing or altering external electromagnetic radiation (radiodensity) resulting in decreased exposure on an electromagnetic waves detector. While, the pluralities of adhesive peptides or peptidomimetics bind to their corresponding receptors of cell-adhesion molecules, preferentially to integrins. These pluralities of adhesive peptides or peptidomimetics comprise cell-adhesive domains that are sufficient for cell membrane binding, wherein a minimum amino-acid sequence of said cell-adhesive domains retains the property of cell adhesion. Whereas, the subcoat of linkers can comprise a biodegradable layer of fluidic material to which the pluralities of adhesive peptides or peptidomimetics are tethered, whereby the pluralities of adhesive peptides or peptidomimetics retain unrestricted mobility, efficient clustering, and redistribution. The MARCs can be nanospheres with isometric dimensions or nanoparticles with anisometric dimensions. Furthermore, the pluralities of adhesive peptides or peptidomimetics can be identical resulting in a monospecific MARC, or different resulting in a polyspecific MARC.

The method of producing the proposed ARCs/MARCs can comprise either tethering, with or without a linker, one or more arms of adhesive peptides (cell-adhesive motifs) or peptidomimetics to a nucleus of radiodense element or molecule, introduction of a radiodense element or molecule to the molecular structure of an adhesive peptide (cell-adhesive domains) or peptidomimetic itself by substitution or addition, or coating a nanocore of radiodense element or molecule with a coat of pluralities of adhesive peptides or peptidomimetics with or without a subcoat of linkers. The adhesive peptides can be produced via serial nucleophilic addition-elimination reactions among the basic amino acid components of said adhesive peptides to form amide bonds or digestion of an adhesive peptide-containing protein. Whereas, adhesive peptidomimetics can arise either from modification of existing adhesive peptides or by designing similar systems that mimic the adhesive peptides, such as peptoids and β-peptides.

The method of using the proposed ARCs/MARCs to help a recipient in need thereof comprises direct injection of the ARCs into an artery using a suitable catheter so that a concomitant therapeutic antiplatelet effect and a diagnostic real-time continuous visualization of said artery can be obtained. These dual therapeutic and diagnostic actions are advantageous during procedural real-time angiography with or without percutaneous intervention, wherein the therapeutic action of said ARCs can be further escalated via manipulation of the structural skeleton of the ARC by incorporation of one or more additional therapeutically active molecule, for example, the vasodilator nitrate ester.

In the preferred embodiment; the ARC comprises an iodine-containing molecule, representing the radiocontrast nucleus, which is bound, with or without a linker, to one or more adhesive arms of cell-adhesive peptide motifs which bind preferentially to the membrane-spanning integrins. The radiocontrast nucleus is preferably a low-osmolarity, non-ionic iodinated molecule linked by a covalent bond to one or more RGD tripeptides representing the adhesive arms. RGD-peptidomimetics, either natural or synthetic, can replace the RGD tripeptide in the structure of the preferred embodiment of the proposed ARCs. For example, the RGD-peptidomimetics can comprise the RYD sequence (Alon, Bayer, and Wilchek 1990) and/or KGD sequence (McLane et al. 2004), and/or other minimum sequence motifs such as the GFOGER amino acid sequence derived from collagen-1 (Knight et al. 2000), the YGISR sequence (Boateng et al. 2005), and A5G81 sequence (Zhu et al. 2018), both derived from laminin.

The method of preparation of the preferred embodiment comprises linking one or more adhesive arms of integrin-binding peptide motifs to an iodinated radiodense nucleus. The adhesive arms of integrin-binding peptide motifs can be produced via serial nucleophilic addition-elimination reactions among the basic amino acid components of said peptide motif to form amide bonds or digestion of an integrin-binding peptides-containing protein. Then, the resultant adhesive arm of integrin-binding peptides is covalently attached to the iodinated radiodense nucleus (for example via condensation reaction of dehydration in the preferred embodiment).

The mechanism of action of the preferred embodiment involves increasing the adsorptive contact time of the radiographic dye to the endothelial layer of the arterial wall, wherein the tripeptide RGD, or the RGD-peptidomimetics, bind preferentially to its corresponding receptor of cell adhesion molecules (CAMs) in the form of the membrane-spanning integrins; while the radiodense iodinated nucleus absorbs or alters external electromagnetic radiation resulting in decreased exposure on an electromagnetic waves detector. Iodine is valuable in the structure of the preferred embodiment because it has a particular advantage as a contrast element for the reason that its innermost electron (k-shell) binding energy is 33.2 keV, similar to the average energy of X-rays used in diagnostic radiography (Sorenson and Phelps 1987). When the incident X-ray energy is closer to the K-edge of the atom it encounters, photoelectric absorption is more likely to occur. The radiodensity of iodinated contrast is 25-30 Hounsfield units per milligram of iodine per milliliter at a tube voltage of 100-120 kVp (Bae 2010).

In a basic exemplifying interaction, integrins are α/β heterodimeric cell adhesion receptors that consist of a bilobular head and two legs that span the plasma membrane (Hynes 2002). The tripeptide RGD is recognized by a subset of integrins including αvβ3, α5β1, and αIIbβ3 (Ruoslahti 1988), wherein the RGD motif binds the integrin head: Arg (arginine) contacts the propeller domain of the α-subunit, and Asp (aspartate) binds the βA domain of the β-subunit at a metal-ion-dependent-adhesion-site (MIDAS) via Mg⁺² or Mn⁺² (Xiong et al. 2002). Two regulatory Ca⁺² cations at the ligand-associated metal-binding site (LIMBS), or synergistic metal-binding site (SyMBS) and adjacent to MIDAS (ADMIDAS), flank the MIDAS metal ion. The Ca⁺² cation at ADMIDAS links the N- and C-terminal helices, stabilizing the inactive conformation (Mould et al. 2003). Accordingly, the adhesive portion (arms) of the proposed ARCs is capable of increasing the adsorptive contact time of the radiodense nucleus to the wall of the target artery.

Likewise, upon platelet activation, both ADP and TXA2 are secreted, in autocrine and paracrine signaling models, to bind their respective receptors on platelet surfaces, whereby a signaling cascade results in an increase in intracellular calcium. Henceforward, the calcium upsurge triggers the calcium-dependent association of gpIIb and gpIIIa to form the activated membrane receptor complex αIIbβ3, which is capable of binding fibrinogen, resulting in platelet aggregation (Calvete 1995). In the preferred embodiment, the adhesive arm of the proposed ARCs comprises the cell-adhesive peptide motif RGD tripeptide. Said peptide motif is recognized by the αIIbβ3 integrin receptor on the cell membrane of platelets, so that competitively inhibits the interaction between the activated αIIbβ3 integrin receptor and its wild-type protein-ligand of fibrinogen, leading to inhibition of platelet aggregation and thrombus formation. Consequently, the previously-mentioned competitive inhibition leads to the advantageous antiplatelet therapeutic action of the proposed ARCs.

The basic advantage of the proposed ARCs/MARCs is the augmented adsorptive contact time to the arterial wall after direct injection of the constructs into the target artery/arterial tree using a suitable catheter, resulting in a real-time continuous background radiographic marking (illumination) of said target artery/arterial tree during procedural angiography with or without percutaneous intervention. This illumination further leads to (i) precise and delicate manipulation of the guidewire which decreases the possibility of developing dangerous arrhythmias, and (ii) dissection or perforation of the coronary artery, which can result from faulty manipulation of the guidewire. (iii) Facilitating intra-operative identification of a myocardial bridge, which appears as a myocardial bite of the dyed coronary artery after injection of the proposed ARCs/MARCs. (iv) Reduction of dye injections diminishes the probability of contrast-induced nephropathy and thyroid dysfunction. (v) Precise and delicate manipulation of the guiding catheter, in the aorta, or the guidewire, in the coronaries, reduces the possibility of an atheromatous plaque rupture and consequential endovascular occlusion and infarction.

Five additional strategic advantages of proposed ARCs/MARCs include: (i) The intervention procedure becomes less operator-dependent, wherein the interventionist is capable of easily manipulating the dense radiopaque coronary guidewire in relation to a radiographically illuminated fainter arterial wall. This issue is critical in the primary percutaneous intervention (PCI) because many patients receive thrombolytic therapy for ST-elevation myocardial infarction (STEMI) or complicated non-ST-elevation myocardial infarction (NSTEMI) because of the unavailability of specialized centers or trained interventionists within 120 minutes duration according to the latest guidelines (Ibanez et al. 2018). A less complicated procedure will enable the expansion of the availability of the primary PCI to wider communities, especially the rural areas, resulting in effective and efficient reperfusion therapy for patients with acute STEMI or complicated NSTEMI. (ii) The proposed ARCs/MARCs are therapeutically active because they inhibit platelet aggregation with a similar mechanism to Glycoprotein IIb/IIIa inhibitors (such as tirofiban, eptifibatide, and abciximab) via inhibiting the interaction of fibrinogen with the integrin glycoprotein (GP) IIb/IIIa on human platelets. This therapeutic action is beneficial in situations of high thrombotic burden and also prevention of during/post-Cath (catheterization) acute intracoronary thrombosis. (iii) The flexible structure can attain additional multiple therapeutic functions via conjugating one or more therapeutically active molecules. For example in the preferred embodiment, a nitrate ester (a vasodilator prodrug) can be conjugated to the structural skeleton of the proposed ARCs/MARCs to result in an additional therapeutic effect of vasodilatation which decreases arterial spasm precipitated by manipulation during interventions. (iv) The advantageous diagnostic action of the proposed ARCs/MARCs, with continuous real-time visualization (temporal extension of fainter dyeing effect) of the arterial tree post-injection (the illumination phase), can enable real-time detection of perfusion efficacy of the myocardium. Therefore, the perfusion defects associated with critical stenoses, or total occlusions (chronic or acute) of the epicardial coronaries can be established in real-time during procedural angiography with or without percutaneous intervention. (v) The proposed ARCs/MARCs can radiographically highlight vulnerable or ulcerative atheromas that are pending rupture. These atheromas are extremely dangerous as they may proceed to acute arterial occlusion, resulting in myocardial infarction or stroke, by activating the coagulation pathway and induction of platelet aggregation. The ulcerative atheroma has exposed, activated integrins due to active inflammation, so the proposed ARCs/MARCs heavily interact and intensely adhere to these activated integrins because the binding affinity of the adhesive portion of the ARCs/MARCs increases 6-10 folds upon interaction with activated integrins, whereas the binding of said adhesive portion is usually a low background in physiological concentrations of Ca⁺² and Mg⁺² (Van Agthoven et al. 2014). The binding affinity, and thus the radiographic density, of the adhesive portion of the ARCs/MARCs, is augmented in the presence of Mn⁺² cations which activate integrins.

### SUMMARY OF THE DRAWINGS

FIG. 1 describes basic structures of multiple embodiments of the proposed adsorptive radiocontrast constructs (ARCs).

FIG. 2 describes a basic method of preparation of the proposed adsorptive radiocontrast constructs (ARCs).

FIG. 3 describes a structure of a monovalent embodiment of the proposed adsorptive radiocontrast constructs (ARCs).

FIG. 4 describes a linker-containing structure of a monovalent embodiment of the proposed adsorptive radiocontrast constructs (ARCs).

FIG. 5a describes a vasodilator-incorporated structure of a linker-containing monovalent embodiment of the proposed adsorptive radiocontrast constructs (ARCs).

FIG. 5b describes a different vasodilator-incorporated structure of a linker-containing monovalent embodiment of the proposed adsorptive radiocontrast constructs (ARCs).

FIG. 6 describes an iodinated-linker structure of a monovalent embodiment of the proposed adsorptive radiocontrast constructs (ARCs).

FIG. 7 describes a structure of a monospecific bivalent embodiment of the proposed adsorptive radiocontrast constructs (ARCs).

FIG. 8 describes a structure of a polyspecific bivalent embodiment of the proposed adsorptive radiocontrast constructs (ARCs).

FIG. 9 describes a structure of a monospecific trivalent embodiment of the proposed adsorptive radiocontrast constructs (ARCs).

FIG. 10 describes a structure of a monospecific quadrivalent embodiment of the proposed adsorptive radiocontrast constructs (ARCs).

FIG. 11 describes two structures of the proposed adsorptive radiocontrast constructs (ARCs) that comprise different radiodense nuclei.

FIG. 12 describes the iodination of an adhesive peptidomimetic representing an embodiment of a basic method of preparation of the proposed adsorptive radiocontrast constructs (ARCs).

FIG. 13 describes a cyclic structure of an iodinated adhesive peptide representing a monovalent embodiment of the proposed adsorptive radiocontrast constructs (ARCs).

FIG. 14 describes a structure of two linked iodinated adhesive peptidomimetics representing a monospecific bivalent embodiment of the proposed adsorptive radiocontrast constructs (ARCs).

FIG. 15 describes a structure of two linked different iodinated adhesive peptides representing a polyspecific bivalent embodiment of the proposed adsorptive radiocontrast constructs (ARCs).

FIG. 16 describes a structure of four linked iodinated adhesive peptidomimetics representing a monospecific quadrivalent embodiment of the proposed adsorptive radiocontrast constructs (ARCs).

FIG. 17 describes a structure of four linked different iodinated adhesive peptides representing a polyspecific quadrivalent embodiment of the proposed adsorptive radiocontrast constructs (ARCs).

FIG. 18a describes a vasodilator-containing structure of an iodinated adhesive peptidomimetic representing a monovalent embodiment of the proposed adsorptive radiocontrast constructs (ARCs).

FIG. 18b describes a vasodilator-containing structure of two linked iodinated adhesive peptidomimetics representing a monospecific bivalent embodiment of the proposed adsorptive radiocontrast constructs (ARCs).

FIG. 19 depicts a schematic description of the interaction of the proposed adsorptive radiocontrast constructs (ARCs) with the arterial wall at the microscopic level combined with the corresponding result at the macroscopic level.

FIG. 20 describes a basic method of preparation of the proposed multivalent adsorptive radiocontrast nanoconstructs (MARCs).

FIG. 21a depicts a schematic description showing the diblock structure of the proposed multivalent adsorptive radiocontrast nanoconstructs (MARCs).

FIG. 21b depicts a schematic description showing the triblock structure of the proposed multivalent adsorptive radiocontrast nanoconstructs (MARCs).

FIG. 22 depicts a schematic description of the interaction of the proposed multivalent adsorptive radiocontrast nanoconstructs (MARCs) with the arterial wall at the microscopic level combined with the corresponding result at the macroscopic level.

### DETAILED DESCRIPTION OF THE DRAWINGS

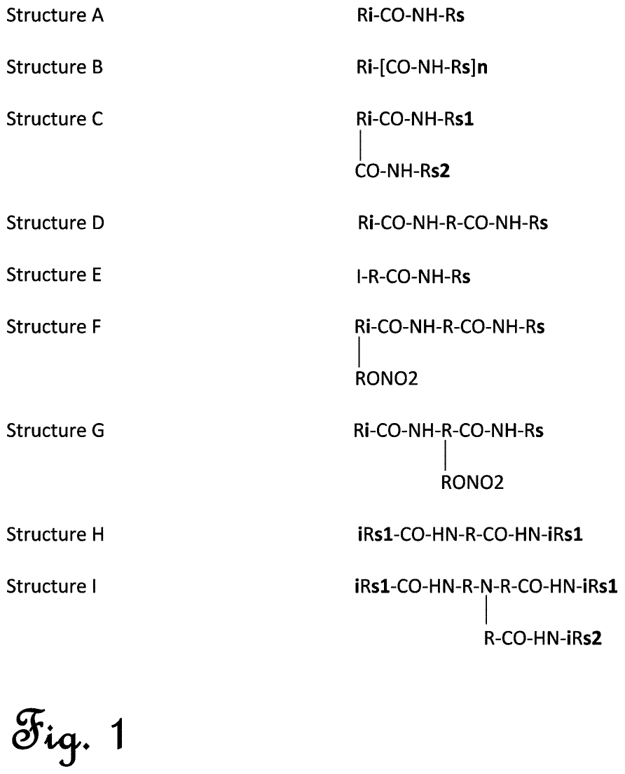

Referring to the drawings in more detail; which describe different embodiments of the structure, synthesis, usage, and mechanism of action of the proposed ARCs. FIG. 1 describes different embodiments of the ARCs basic structure. Structure A depicts a simple structure of the ARCs, wherein an iodinated functional group (Ri), representing the radiodense nucleus, is tethered to a cell-adhesive peptide motif or peptidomimetic (Rs), representing the adhesive arm, via an amide bond. Structure B depicts a multivalent structure of the proposed ARCs, wherein an iodinated functional group (Ri), representing the radiodense nucleus, is tethered to an (n) number of cell-adhesive peptide motifs or peptidomimetics (Rs), representing the adhesive arms, via amide bonds. Structure C depicts a polyspecific bivalent structure of the proposed ARCs, wherein an iodinated functional group (Ri), representing the radiodense nucleus, is tethered to two different cell-adhesive peptide motifs or peptidomimetics (Rs1 and Rs2), representing two different adhesive arms, via amide bonds. Structure D depicts a monovalent structure of the proposed ARCs with a linker, wherein an iodinated functional group (Ri), representing the radiodense nucleus, is tethered via a linker (R) to a cell-adhesive peptide motif or peptidomimetic (Rs), representing the adhesive arm via amide bonds. Structure E depicts a monovalent structure of the proposed ARCs with a linker, wherein iodine (I), representing the radiodense nucleus, is covalently bound to a linker (R) which—in turn—is tethered to a cell-adhesive peptide motif or peptidomimetic (Rs), representing the adhesive arms via an amide bond. Structure F depicts a monovalent structure of the proposed ARCs with a linker, wherein a nitrate ester, representing an additional therapeutically active molecule, is tethered to an iodinated functional group (Ri), representing the radiodense nucleus, which—in turn—is tethered via a linker (R) to a cell-adhesive peptide motif or peptidomimetic (Rs), representing the adhesive arm via amide bonds. Structure G depicts a monovalent structure of the proposed ARCs with a linker, wherein a nitrate ester, representing an additional therapeutically active molecule, is tethered to the linker (R) which links between an iodinated functional group (Ri), representing the radiodense nucleus, and a cell-adhesive peptide motif or peptidomimetic (Rs) representing the adhesive arm. Structure H depicts a monospecific bivalent structure of the proposed ARCs with a linker, wherein said linker (R) tethers two iodinated cell-adhesive domains (iRs), representing the adhesive peptides or peptidomimetics, that contain within its molecular structure a radiodense element or molecule. Structure I depicts a polyspecific trivalent structure of the proposed ARCs with a branched linker, wherein said branched linker (NR3) tethers three different iodinated cell-adhesive domains (iRs1 and iRs2) representing the adhesive peptides or peptidomimetics that contain within its molecular structure a radiodense element or molecule.

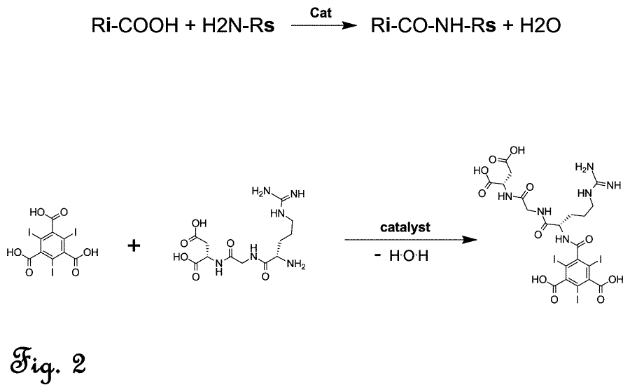

FIG. 2 describes a basic method of preparation of the proposed ARCs, wherein an iodinated molecule (herein an iodinated carboxylic acid, Ri-COOH) is reacting with a cell-adhesive domain (H2N-Rs) using a catalyst resulting in an ARC, which comprises an iodinated radiodense nucleus linked to an adhesive arm, and a water molecule.

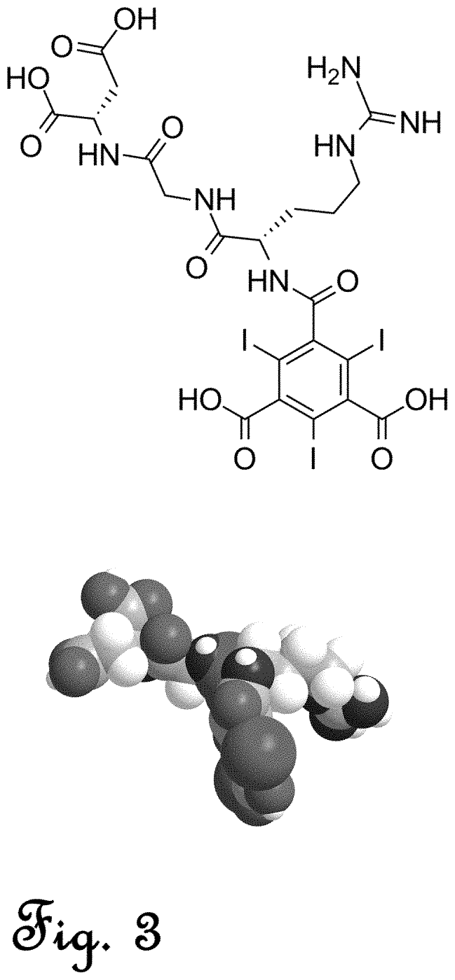

FIG. 3 describes a structure of a monovalent embodiment of the proposed ARCs. The figure depicts the skeletal formula followed by the space-filling calotte model below. As shown, the monovalent ARC comprises a radiodense nucleus, comprising an iodine-containing molecule, covalently bound to an adhesive arm comprising the cell-adhesive peptide motif of integrin-binding RGD tripeptide. The advantages of this structure comprise the aimed dual therapeutic and diagnostic actions, wherein the cell-adhesive peptide motif of integrin-binding RGD tripeptide can adsorb to the endothelial layer of the arterial wall, especially if diseased since the adsorption capacity is directly proportional to the disease state of the arterial wall, for longer duration post-injection via a suitable catheter, so that the iodine-containing radiodense nucleus continuously marks the arterial wall during a digitalized X-ray based real-time visualization. This diagnostic action is advantageous during real-time angiography, especially if a percutaneous intervention is performed. Additionally, the cell-adhesive peptide motif of integrin-binding RGD tripeptide competitively interacts with its corresponding receptor of αIIbβ3 which is a highly abundant heterodimeric platelet receptor that usually binds to fibrinogen with augmented affinity upon platelet activation. This therapeutic antiplatelet action is advantageous during percutaneous intervention, especially in the case of (I) primary intervention during acute myocardial infarction to prevent the no-reflow phenomenon, (II) insufficient patient loading with an antiplatelet drug prior to PCI, and (III) heavy thrombus burden during intervention.

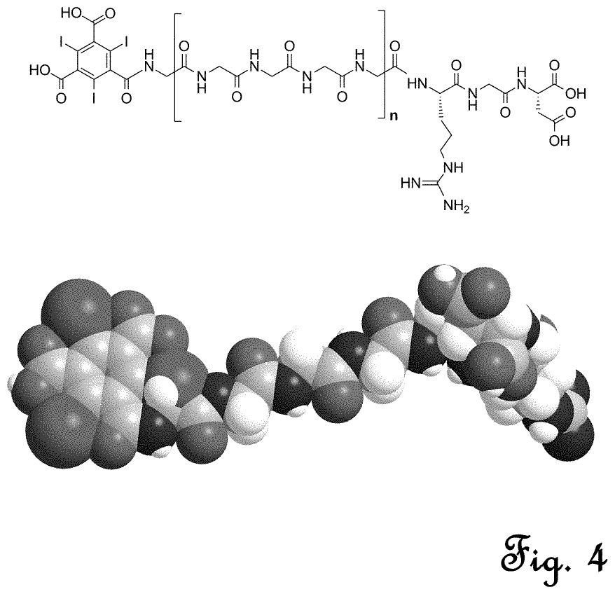

FIG. 4 describes a linker-containing structure of a monovalent embodiment of the proposed ARCs, wherein a linker is integrated between an iodine-containing radiodense nucleus and an adhesive arm. The figure depicts the skeletal formula followed by the space-filling calotte model below. As shown, the iodine-containing radiodense nucleus is linked, via a linker formed of an (n) number of flexible glycine (G) sequence(s), to an adhesive arm comprising the cell-adhesive peptide motif of integrin-binding RGD tripeptide. The advantages of this structure comprise the previously mentioned dual therapeutic and diagnostic actions, in addition to the molecular flexibility and spacing provided by the flexible linker, so that decreasing the steric hindrance by improving the spatial arrangement of the proposed ARCs during interaction with their corresponding receptors of cell-adhesion molecules.

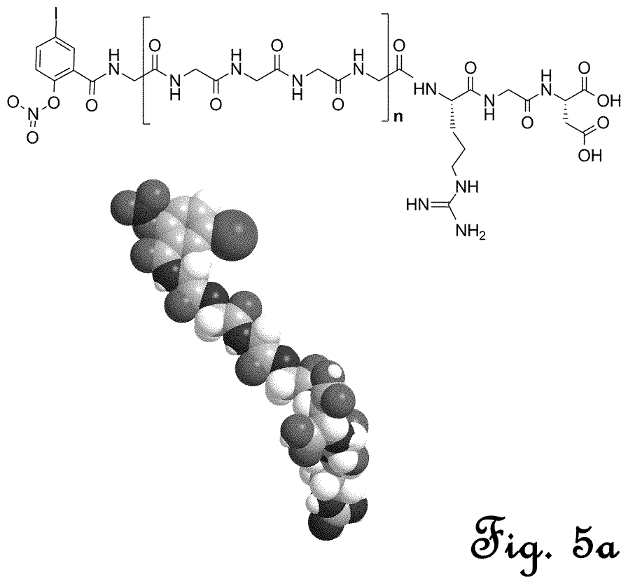

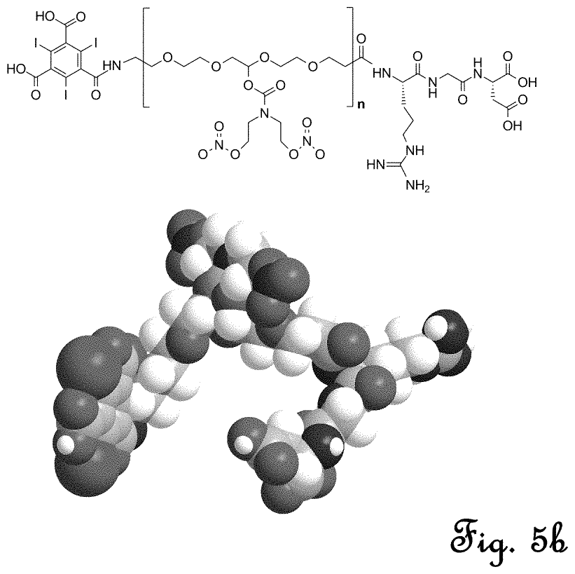

FIG. 5a describes a vasodilator-incorporated structure of a linker-containing monovalent embodiment of the proposed ARCs, wherein a nitrate ester (a vasodilator prodrug), representing an additional therapeutically active molecule, is tethered to a radiodense nucleus containing iodine, which—in turn—is tethered via a linker to an adhesive arm of cell-adhesive peptide motif or peptidomimetic. The figure depicts the skeletal formula followed by the space-filling calotte model below. As shown, the nitrate ester-containing iodinated radiodense nucleus is linked, via a linker formed of an (n) number of flexible glycine (G) sequence(s), to an adhesive arm comprising the cell-adhesive peptide motif of integrin-binding RGD tripeptide. The advantages of this structure comprise the previously mentioned dual therapeutic and diagnostic actions, besides an additional therapeutic effect of vasodilatation which decreases arterial spasm precipitated by manipulation during interventions. Likewise, the composite nature of the structure of the proposed ARCs can permit a supplementary therapeutic effect that can be obtained via therapeutically active metabolites of said ARCs (herein, traces of the antiplatelet/anti-inflammatory salicylic acid). Moreover, the proposed structure retains the molecular flexibility and spacing provided by the flexible linker, decreasing the steric hindrance by improving the spatial arrangement of the ARCs during interacting with their corresponding receptors of cell-adhesion molecules.

FIG. 5b describes a different vasodilator-incorporated structure of a linker-containing monovalent embodiment of the proposed ARCs, wherein a nitrate ester (a vasodilator prodrug), representing an additional therapeutically active molecule, is anchored to a linker that tethers a radiodense nucleus and an adhesive arm. The figure depicts the skeletal formula followed by the space-filling calotte model below. As shown, the nitrate ester-containing PEG (polyethylene glycol) linker tethers a radiodense nucleus comprising an iodinated molecule to an adhesive arm comprising the cell-adhesive peptide motif of integrin-binding RGD tripeptide. The advantages of this structure comprise the previously mentioned dual therapeutic and diagnostic actions, besides an additional therapeutic effect of vasodilatation which decreases arterial spasm precipitated by manipulation during interventions. Also, the proposed structure retains the molecular flexibility and spacing provided by the PEG linker, so that decreasing the steric hindrance.

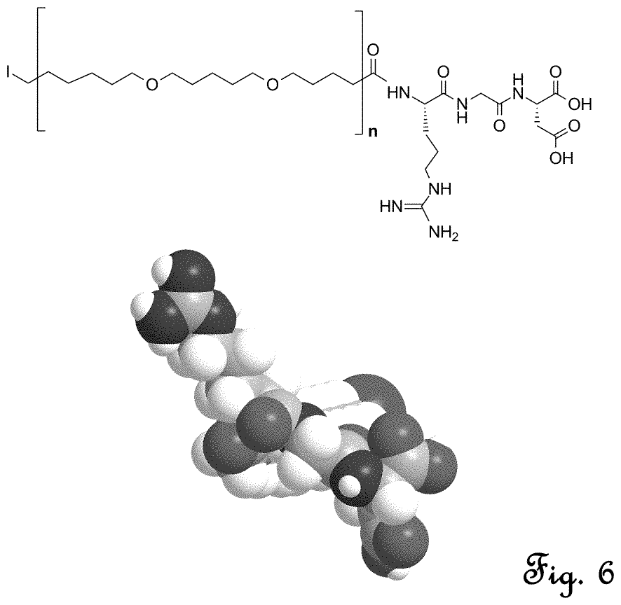

FIG. 6 describes a linker-containing structure of a monovalent embodiment of the proposed ARCs, wherein a linker is integrated between a radiodense nucleus of elemental iodine and an adhesive arm. The figure depicts the skeletal formula followed by the space-filling calotte model below. As shown, an iodinated PEG linker is tethered to an adhesive arm comprising the cell-adhesive peptide motif of integrin-binding RGD tripeptide. The advantages of this structure retain the previously mentioned dual therapeutic and diagnostic actions, in addition to the molecular flexibility and spacing provided by the flexible linker, so that decreasing the steric hindrance by improving the spatial arrangement of the ARCs during interaction with their corresponding receptors of cell-adhesion molecules. As well as, replacing the bulky radiodense nucleus of an iodinated molecule with elemental iodine tethered directly to the linker, further reduces the steric hindrance.

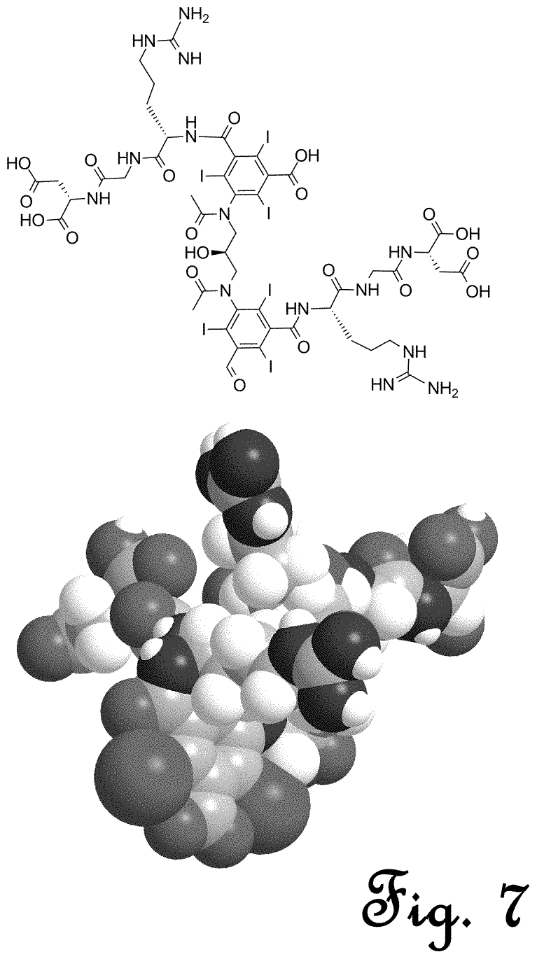

FIG. 7 describes a structure of a monospecific bivalent embodiment of the proposed ARCs. The figure depicts the skeletal formula followed by the space-filling calotte model below. As shown, the monospecific bivalent ARC comprises a radiodense nucleus of an iodine-containing molecule covalently bound to two adhesive arms comprising the cell-adhesive peptide motif of integrin-binding RGD tripeptides. The advantages of this structure retain the previously mentioned dual therapeutic and diagnostic actions, but with an augmented functional affinity that is achieved by the bivalency.

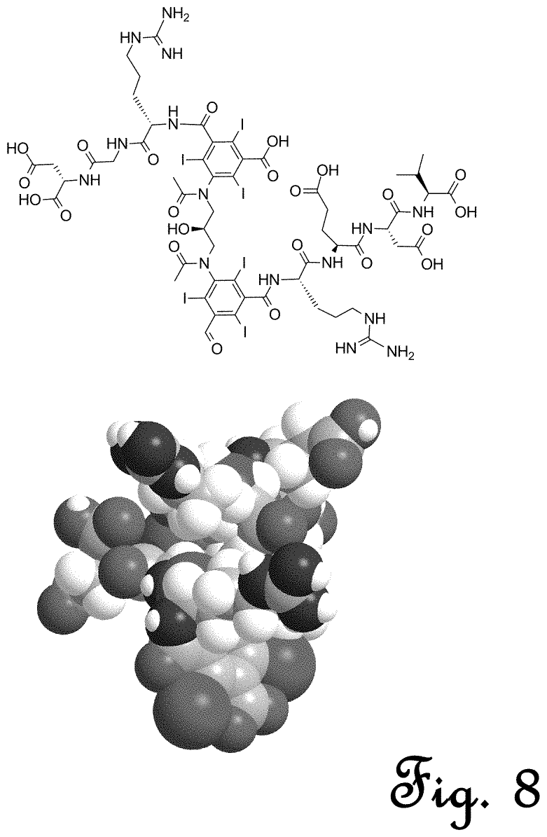

FIG. 8 describes a structure of a polyspecific bivalent embodiment of the proposed ARCs. The figure depicts the skeletal formula followed by the space-filling calotte model below. As shown, the polyspecific bivalent ARC comprises an iodine-containing radiodense nucleus covalently bound to two adhesive arms comprising two integrin-binding cell-adhesive peptide motifs of RGD tripeptide and REDV quadripeptide. The advantages of this structure retain the previously mentioned dual therapeutic and diagnostic actions, but with an augmented functional affinity that is achieved by the bivalency. Additionally, the polyspecificity expands the pool of corresponding receptors available for the ARC, wherein in the provided embodiment of FIG. 8, the RGD tripeptide has an augmented affinity for α3β1, α5β1, α8β1, αvβ1, αvβ3, αvβ5, αvβ6, αIIbβ3 integrin receptors, while the REDV quadripeptide has an augmented affinity for α4β1 integrin receptor.

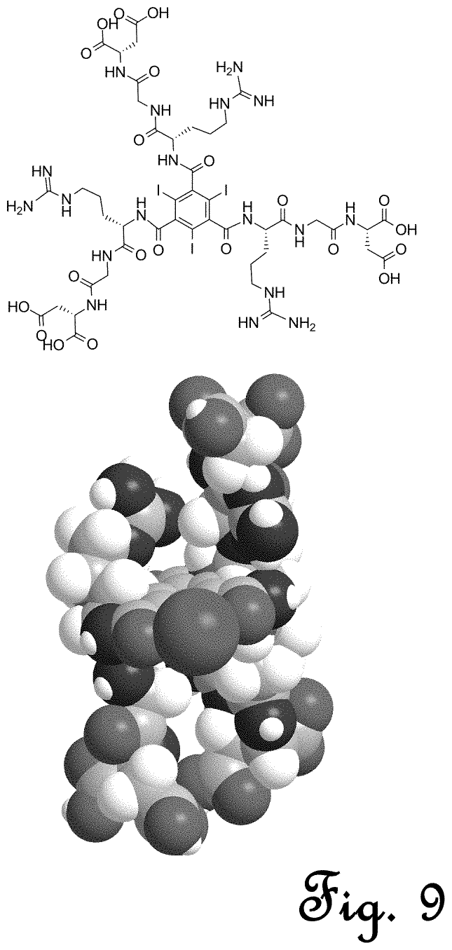

FIG. 9 describes a structure of a monospecific trivalent embodiment of the proposed ARCs. The figure depicts the skeletal formula followed by the space-filling calotte model below. As shown, the monospecific trivalent ARC comprises an iodine-containing radiodense nucleus covalently bound to three adhesive arms comprising three integrin-binding cell-adhesive peptide motifs of RGD tripeptides. The advantages of this structure retain the previously mentioned dual therapeutic and diagnostic actions, but with the multivalency-related augmented functional affinity that is achieved by the trivalency.

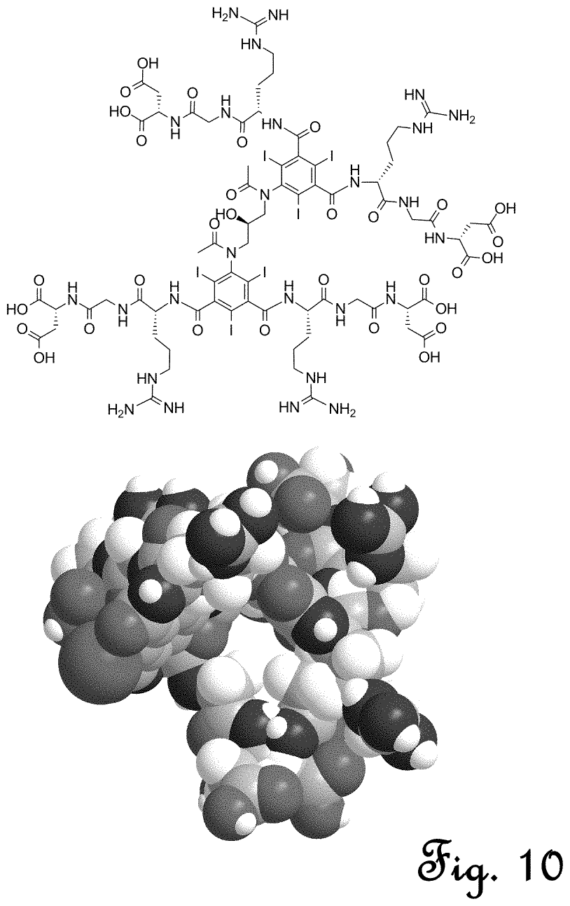

FIG. 10 describes a structure of a monospecific quadrivalent embodiment of the proposed ARCs. The figure depicts the skeletal formula followed by the space-filling calotte model below. As shown, the monospecific quadrivalent ARC comprises an iodine-containing radiodense nucleus covalently bound to four adhesive arms comprising four integrin-binding cell-adhesive peptide motifs of RGD tripeptides. The advantages of this structure retain the previously mentioned dual therapeutic and diagnostic actions, but with an augmented functional affinity that is achieved by the quadrivalency, as an example of multivalency.

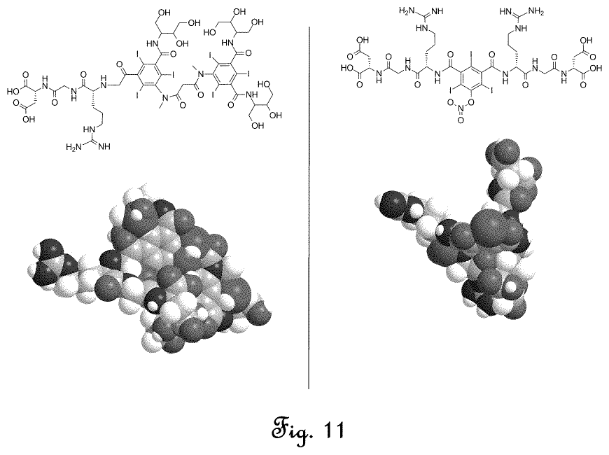

FIG. 11 describes two different structures of the ARCs wherein the radiodense nucleus is different in each structure. For each structure, the figure depicts the skeletal formula followed by the space-filling calotte model below it. As shown in each structure, the ARC comprises an iodine-containing radiodense nucleus covalently bound to one or more adhesive arms comprising integrin-binding cell-adhesive peptide motifs of RGD tripeptides. These structures aim to emphasize the variability of the radiodense nucleus that can be used in the structure of the proposed ARCs, providing that they achieve the condition of radiodensity. And likewise, variable cell-adhesive peptide motifs, or peptidomimetics, can be used as adhesive arms in the structure of the proposed ARCs, as long as they achieve the condition of adsorptive/adhesive fixation.

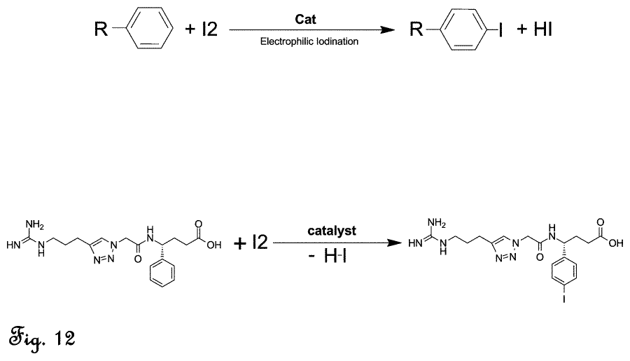

FIG. 12 describes a basic method of preparation of the proposed ARCs, wherein the iodination of an adhesive peptide/peptidomimetic represents another embodiment of such method of preparation. As shown, an adhesive peptide/peptidomimetic (herein 1-R benzene, R-C6H5), comprising a cell-adhesive domain, is reacting with iodine (12) using a catalyst in an electrophilic iodination reaction to produce an ARC that comprises an adhesive peptide/peptidomimetic that contains within its molecular structure a radiodense element (herein, iodine) and hydrogen iodide.

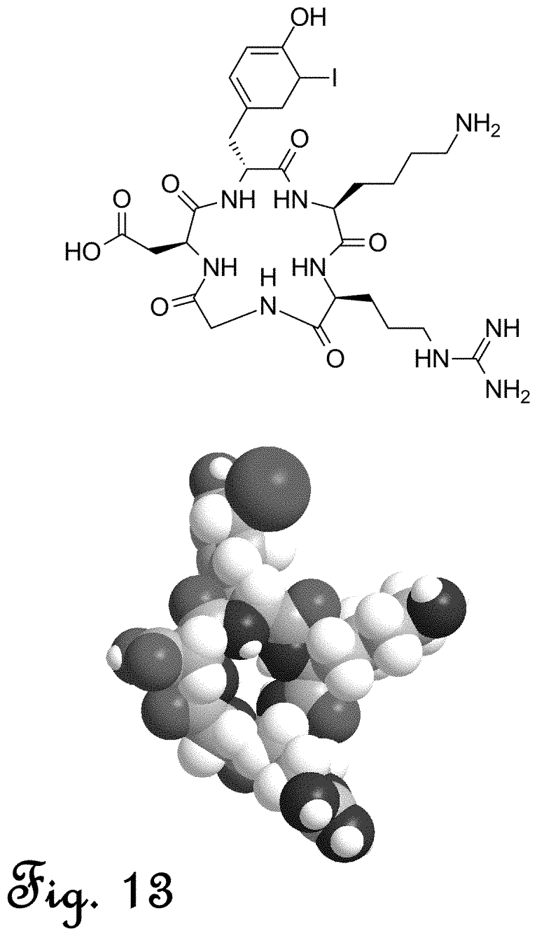

FIG. 13 describes the cyclic structure of an iodinated adhesive peptide representing a monovalent embodiment of the proposed ARCs. The figure depicts the skeletal formula followed by the space-filling calotte model below. As shown, the monovalent ARC comprises an iodine-containing adhesive peptide comprising the integrin-binding cell-adhesive domain of the iodinated cyclic RGDyK peptide. The advantages of this structure retain the previously mentioned dual therapeutic and diagnostic actions, but with augmented stability and affinity that are achieved by the cyclic structure.

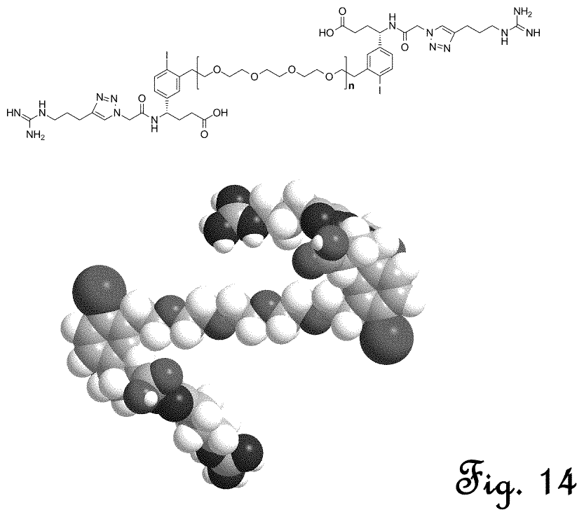

FIG. 14 describes a structure of two linked iodinated adhesive peptidomimetics representing a monospecific bivalent embodiment of the proposed ARCs, wherein a linker is integrated between two iodinated adhesive peptidomimetics to form a flexible ARC. The figure depicts the skeletal formula followed by the space-filling calotte model below. As shown, the monospecific bivalent ARC comprises two linked (via a PEG linker) iodine-containing adhesive peptidomimetics comprising the integrin-binding cell-adhesive domain of iodinated RGD-peptidomimetics. The advantages of this structure retain the previously mentioned dual therapeutic and diagnostic actions, but with an augmented functional affinity that is achieved by the flexible bivalency, which—in turn—is achieved via the molecular flexibility and spacing provided by the flexible linker, so that decreasing the steric hindrance by improving the spatial arrangement of the ARCs during interacting with their corresponding receptors of cell-adhesion molecules.

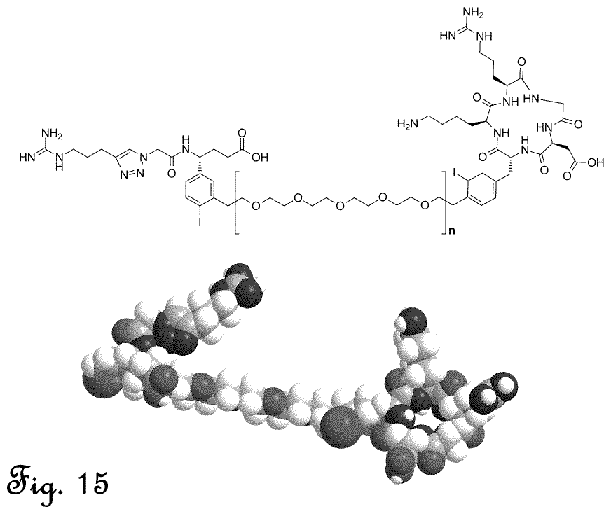

FIG. 15 describes a structure of two linked different iodinated adhesive peptides/peptidomimetics representing a polyspecific bivalent embodiment of the proposed ARCs, wherein a linker is integrated between an iodinated adhesive peptidomimetic and an iodinated adhesive peptide to form a flexible ARC. The figure depicts the skeletal formula followed by the space-filling calotte model below. As shown, the polyspecific bivalent ARC comprises an iodinated adhesive RGD-peptidomimetic linked (via a PEG linker) to an iodine-containing adhesive peptide comprising the integrin-binding cell-adhesive domain of iodinated cyclic RGDyK. The advantages of this structure retain the previously mentioned dual therapeutic and diagnostic actions, but with an augmented functional affinity that is achieved by the flexible bivalency, which—in turn—is achieved via the molecular flexibility and spacing provided by the flexible linker, so that decreasing the steric hindrance. Moreover, the polyspecificity expands the pool of corresponding receptors available for the ARC.

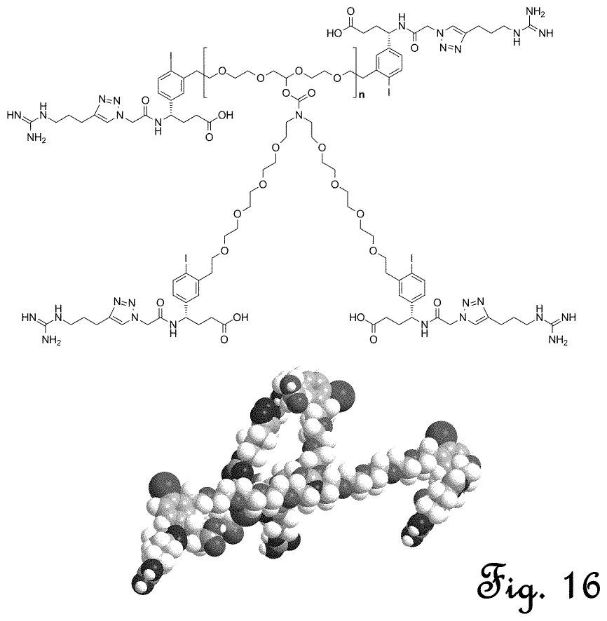

FIG. 16 describes a structure of four linked iodinated adhesive peptidomimetics representing a monospecific quadrivalent embodiment of the proposed ARCs, wherein a branched linker is integrated between four iodinated adhesive peptidomimetics to form a flexible ARC. The figure depicts the skeletal formula followed by the space-filling calotte model below. As shown, the monospecific quadrivalent ARC comprises four linked (via a branched PEG linker) iodine-containing adhesive peptidomimetics comprising the integrin-binding cell-adhesive domain of iodinated RGD-peptidomimetics. The advantages of this structure retain the previously mentioned dual therapeutic and diagnostic actions, but with an augmented functional affinity that is achieved by the flexible quadrivalency, which—in turn—is achieved via the molecular flexibility and spacing provided by the branched flexible linker, so that decreasing the steric hindrance.

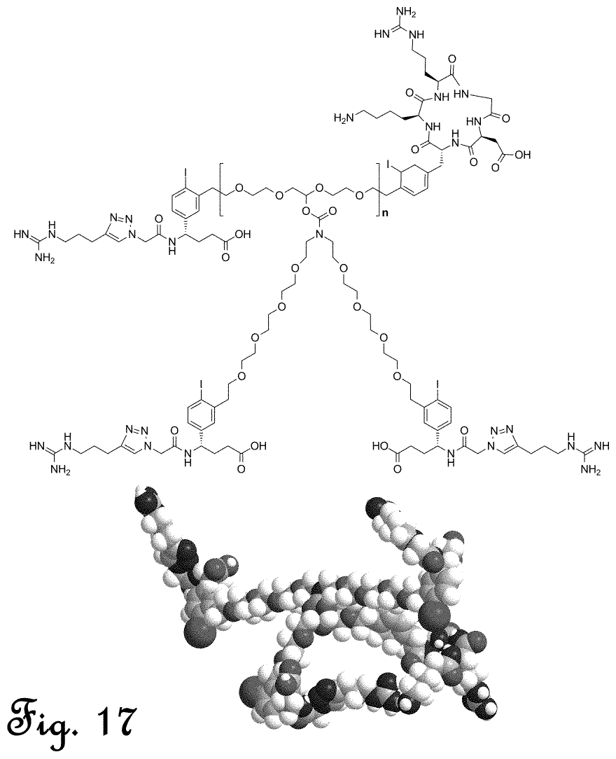

FIG. 17 describes a structure of four linked different iodinated adhesive peptides/peptidomimetics representing a polyspecific quadrivalent embodiment of the proposed ARCs, wherein a branched linker is integrated between three iodinated adhesive peptidomimetics and an iodinated adhesive peptide to form a flexible ARC. The figure depicts the skeletal formula followed by the space-filling calotte model below. As shown, the polyspecific quadrivalent ARC comprises three iodinated adhesive RGD-peptidomimetic linked (via a branched PEG linker) to an iodine-containing adhesive peptide comprising the integrin-binding cell-adhesive domain of iodinated cyclic RGDyK. The advantages of this structure retain the previously mentioned dual therapeutic and diagnostic actions, but with an augmented functional affinity that is achieved by the flexible quadrivalency, which—in turn—is achieved via the molecular flexibility and spacing provided by the branched flexible linker, so that decreasing the steric hindrance. Also, the polyspecificity expands the pool of corresponding receptors available for the ARC.

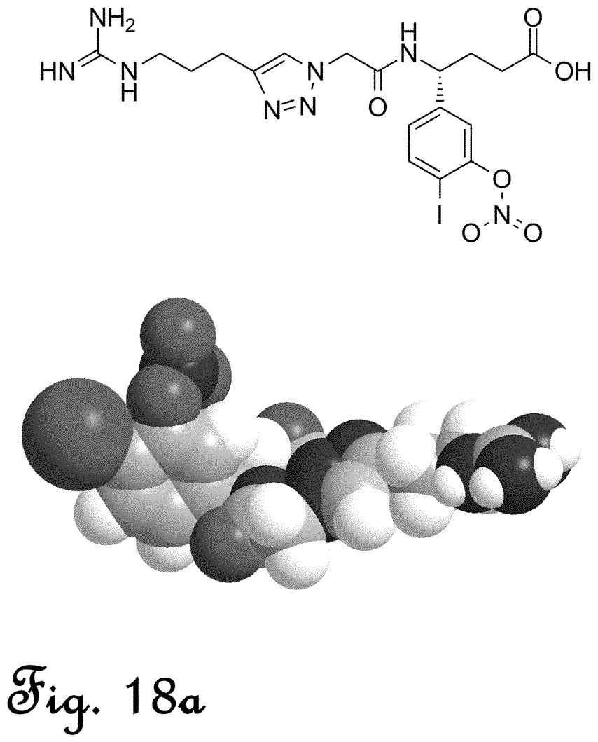

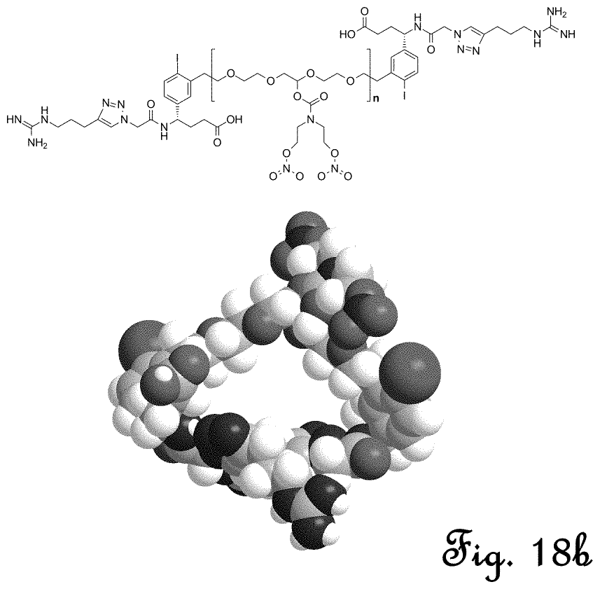

FIG. 18a describes a vasodilator-containing structure of an iodinated adhesive peptidomimetic representing a monovalent embodiment of the proposed ARCs, wherein a nitrate ester (a vasodilator prodrug), representing an additional therapeutically active molecule, is anchored to the molecular structure of the iodinated adhesive peptide/peptidomimetic. The figure depicts the skeletal formula followed by the space-filling calotte model below. As shown, the nitrate ester is tethered to the iodine-containing adhesive peptide comprising the integrin-binding cell-adhesive domain of iodinated RGD-peptidomimetic. The advantages of this structure comprise the previously mentioned dual therapeutic and diagnostic actions, besides an additional therapeutic effect of vasodilatation which decreases arterial spasm precipitated by manipulation during interventions.

FIG. 18b describes a vasodilator-containing structure of two linked iodinated adhesive peptidomimetics representing a monospecific bivalent embodiment of the proposed ARCs, wherein a nitrate ester (a vasodilator prodrug), representing an additional therapeutically active molecule, is anchored to the linker that tethers two iodinated adhesive peptide/peptidomimetics. The figure depicts the skeletal formula followed by the space-filling calotte model below. As shown, the nitrate ester is tethered to the PEG linker that links two iodine-containing adhesive peptidomimetics comprising the integrin-binding cell-adhesive domain of iodinated RGD-peptidomimetics. The advantages of this structure comprise the previously mentioned dual therapeutic and diagnostic actions, besides an additional therapeutic effect of vasodilatation which decreases arterial spasm precipitated by manipulation during interventions. Also, the proposed structure retains the molecular flexibility and spacing provided by the PEG linker, so that decreasing the steric hindrance.

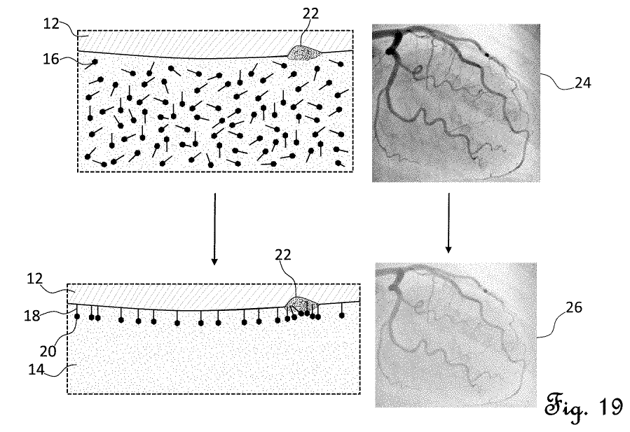

FIG. 19 depicts a schematic description of the basic mechanism of action of the proposed ARCs 16, wherein the interaction of said ARCs 16 with a target cell 12 of the inner arterial wall is shown at the microscopic level, combined with the corresponding result at the macroscopic level. Said target cell 12 presents surface receptors in the form of cell-adhesion molecules (not shown), preferentially integrins, and during the injection phase 24, the ARCs 16 are rushed through the bloodstream 14 due to elevated intra-arterial pressure (normally, 120 mmHg during systole). Then, a subsequent illumination phase 26 follows, wherein a considerable amount of the injected ARCs 16 interacts with the target cells 12 of the inner arterial wall leading to adsorptive/adhesive fixation. This fixation results from the simultaneous interaction between the adhesive arms 18 of each ARC 16 with one or more corresponding receptors of cell adhesion molecules (not shown) presented on each target cell 12. The presence of a heavier density of exposed corresponding receptors in an arterial wall lesion 22 results in effective clustering of the ARCs 16 leading to the highlighting of said lesion 22. The dyeing effect of the targeted artery/arterial tree during both the short injection phase 24 and the longer subsequent illumination phase 26 is achieved via absorption or alternation of external electromagnetic radiation provided by the radiodense nucleus 20 of the proposed ARCs 16. On the macroscopic level, injection phase 24 shows the dyeing effect of intra-arterial injection of the proposed ARCs 16 in the corresponding drawing which depicts the left system of the epicardial coronaries. The dense-dyeing macroscopic injection phase 24 is followed by a prolonged phase of fainter macroscopic illumination 26 which shows the fainter dyeing in the corresponding drawing which also depicts the left system of the epicardial coronaries.

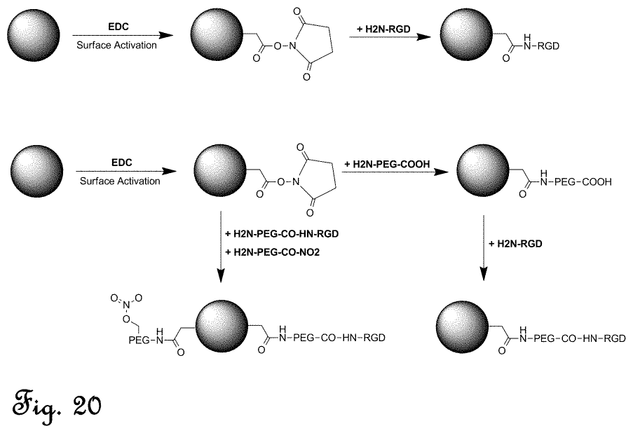

FIG. 20 describes a basic method of preparation of the proposed multivalent adsorptive radiocontrast nanoconstructs (MARCs), wherein a radiodense nanocore undergoes a surface activation, which can be achieved in a preferred embodiment using 1-ethyl-3-(3-dimethylaminopropyl)-carbodiimide (EDC), to generate active esters that is less prone to hydrolysis, followed by in-water coupling with pluralities of adhesive peptides or peptidomimetics, which can be comprising the integrin-binding cell-adhesive domain of RGD tripeptides, to generate multivalent adsorptive radiocontrast nanoconstructs (MARCs) of diblock nanospheres and/or diblock nanoparticles (according to the shape of pre-activation radiodense nanocore). In another embodiment, after surface activation, the step of in-water coupling occurs with a liker, which can be a PEG chain, then said linker is activated to react with pluralities of adhesive peptides or peptidomimetics, which can be comprising the integrin-binding cell-adhesive domain of RGD tripeptides, to generate multivalent adsorptive radiocontrast nanoconstructs (MARCs) of triblock nanospheres and/or triblock nanoparticles. In an additional embodiment, the linker PEG chains can be separately activated and react with: (I) pluralities of adhesive peptides/peptidomimetic, which can be comprising the integrin-binding cell-adhesive domain of RGD tripeptides to form pegylated pluralities of adhesive peptides/peptidomimetic, and (II) one or more additional therapeutically active molecule, herein the vasodilator nitrate esters, to form a pegylated therapeutically active molecule. Then, these pegylated molecules react, in an in-water coupling step, with the already-activated surface of radiodense nanocores to generate multivalent adsorptive radiocontrast nanoconstructs (MARCs) of triblock nanospheres and/or triblock nanoparticles.

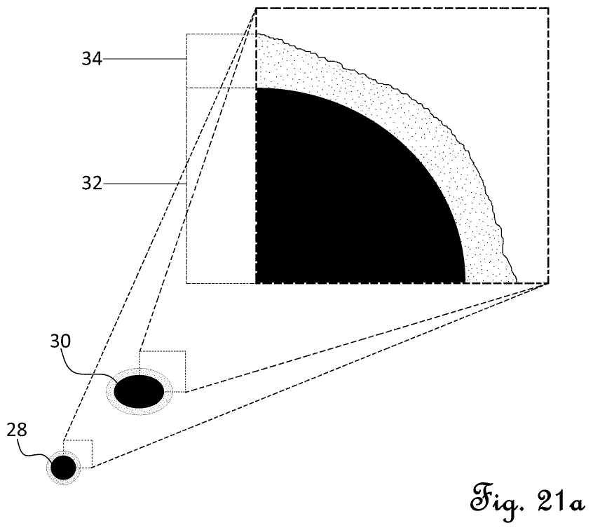

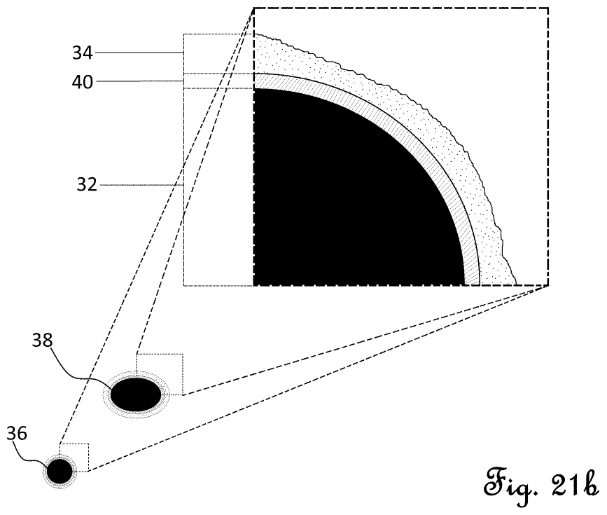

FIG. 21a depicts a schematic description showing the diblock structure of the proposed MARCs. The structure of MARCs of diblock nanospheres 28 and nanoparticles 30 comprises a spherical/non-spherical radiodense nanocore 32, which is capable of absorbing or altering external electromagnetic radiation resulting in decreased exposure on an electromagnetic waves detector, coated with a coat of pluralities adhesive peptides/peptidomimetics 34, whereby these pluralities of adhesive peptides/peptidomimetics 34 are presented to interact simultaneously with multiple corresponding binding sites. The advantages of this structure comprise the previously mentioned dual therapeutic and diagnostic actions.

FIG. 21b depicts a schematic description showing the triblock structure of the proposed MARCs. The structure of MARCs of triblock nanospheres 36 and nanoparticles 38 comprises a spherical/non-spherical radiodense nanocore 32, which is capable of absorbing or altering external electromagnetic radiation resulting in decreased exposure on an electromagnetic waves detector, coated with a subcoat of linkers 40, which can comprise a biodegradable layer of fluidic material above which a coat of pluralities adhesive peptides/peptidomimetics 34 can be incorporated, whereby these pluralities of adhesive peptides/peptidomimetics 34 are presented to interact simultaneously with multiple corresponding binding sites. Said structure increases the ability of dynamic repositioning of these bound pluralities of adhesive peptides/peptidomimetics 34. The advantages of this structure comprise the previously mentioned dual therapeutic and diagnostic actions. Besides, the proposed structure retains the molecular flexibility and spacing provided by the linker subcoat, so that decreasing the steric hindrance.

An additional advantage of the proposed structures is multivalency, wherein the structure helps simultaneous multiple binding interactions between the multiple ligands presented by said constructs with their target receptors. So, individual binding events increase the probability of other interactions occurring between the unbound ligands of said constructs and their corresponding binding sites; due to an increase in the local concentration of each binding ligand in proximity to the corresponding binding site. Individually, each binding interaction may be readily broken; however, when many simultaneous binding interactions are present, the transient unbinding of a single site does not allow the molecule to diffuse away, and the binding of that weak interaction is likely to be restored. Accordingly, the multivalency and/or the linker flexibility with their corresponding augmented functional affinity can compensate for the notable binding weakness of the interaction between isolated domains (herein, cell-adhesive domains), without their complete protein (tertiary) structure, and their corresponding receptors.

So, the multivalency of the proposed constructs results in a stable construct-cell adhesion by increasing the avidity of the ligand-receptor non-covalent binding. Additionally, different ligands can be presented simultaneously by said constructs, so they can be monospecific if the presented ligands are identical, or polyspecific if the presented ligands are non-identical. Besides, the proposed structure is more biomimetic and may be essential for achieving the desired action.

The biodegradable structure is advantageous, wherein proteolytic-cleavable bonds guarantee a controllable degradation of the constructs which can then release still-therapeutically active byproduct molecules, for example, the gradual constructs degradation and wash-up are accompanied by continuous release of therapeutically active antiplatelet adhesive peptides or peptidomimetics comprising αIIbβ3 integrin-binding cell-adhesive domains of RGD tripeptides/RGD-peptidomimetics. Also, the flexible composite structure can attain multiple therapeutic functions via conjugating one or more therapeutically active molecules. Besides, the flexibility provided by the linkers and/or the fluidic nature of the subcoat enables unrestricted mobility of the bound biomolecules facilitating focal adhesion, clustering, and redistribution, which help decrease the steric hindrance and increase the avidity due to increasing the availability of binding ligands to their corresponding receptors. Moreover, the proposed exemplary use of PEG linkers/subcoat increases solubility, decreases immunogenicity and antigenicity, shields the constructs from recognition by the reticuloendothelial system, and inhibits non-specific interaction and opsonization. Additionally, the degree of fluidity of the PEG subcoat can be controlled by monitoring the molecular weight of PEG molecules.

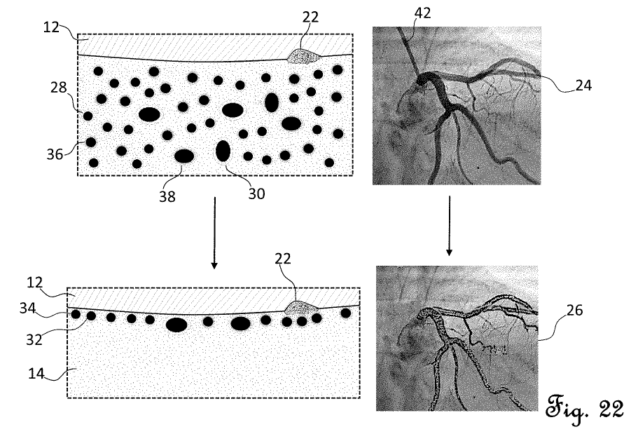

FIG. 22 depicts a schematic description of the basic mechanism of action of the proposed MARCs (herein, MARCs of diblock nanospheres 28 and nanoparticles 30 as well as MARCs of triblock nanospheres 36 and nanoparticles 38), wherein the interaction of said MARCs 28, 30, 36, and 38 with a target cell 12 of the inner arterial wall is shown at the microscopic level, combined with the corresponding result at the macroscopic level. Said target cell 12 presents surface receptors in the form of cell-adhesion molecules (not shown), preferentially integrins, and during the injection phase 24, the MARCs 28, 30, 36, and 38 are rushed through the bloodstream 14 due to elevated intra-arterial pressure (normally, 120 mmHg during systole). Then, a subsequent illumination phase 26 follows, wherein a considerable amount of the injected MARCs 28, 30, 36, and 38 interacts with the target cells 12 of the inner arterial wall leading to adsorptive/adhesive fixation. This fixation results from the simultaneous interaction between the coat of pluralities of adhesive peptides/peptidomimetics 34 of each MARC 28, 30, 36, and 38 with multiple corresponding receptors of cell adhesion molecules (not shown) presented on each target cell 12. The presence of a heavier density of exposed corresponding receptors in an arterial wall lesion 22 results in effective clustering of the MARCs 28, 30, 36, and 38 leading to the highlighting of said lesion 22. The dyeing effect of the targeted artery/arterial tree during both the short injection phase 24 and the longer subsequent illumination phase 26 is achieved via absorption or alternation of external electromagnetic radiation provided by the radiodense nanocore 32 of the proposed MARCs 28, 30, 36, and 38. On the macroscopic level, the injection phase 24 shows the dyeing effect of intra-arterial injection of the proposed MARCs 28, 30, 36, and 38, via a suitable catheter 42, in the corresponding drawing which depicts the left system of the epicardial coronaries. The dense-dyeing macroscopic injection phase 24 is followed by a prolonged phase of fainter macroscopic illumination 26 which shows the fainter dyeing in the corresponding drawing which also depicts the left system of the epicardial coronaries. The figure also shows a method of using the invention to help a recipient in need thereof, wherein a real-time continuous visualization of an artery can be obtained via intra-arterial direct injection of the proposed constructs using a suitable catheter 42. Accordingly, the present invention is advantageous when used for procedural real-time angiography with or without percutaneous intervention.

### DRAWINGS REFERENCE NUMBER

| | | | |
|---|---|---|---|
| 12 | Target cell | 14 | Bloodstream |
| 16 | Adsorptive radiocontrast | 18 | Adhesive arm |
| | construct (ARC) | | |
| 20 | Radiodense nucleus | 22 | Lesion |
| 24 | Injection phase | 26 | Illumination phase |
| 28 | Multivalent adsorptive | 30 | Multivalent adsorptive |
| | radiocontrast nanoconstruct | | radiocontrast nanoconstruct |
| | (MARC) of diblock nano- | | (MARC) of diblock nano- |
| | spheres | | particles |
| 32 | Radiodense nanocore | 34 | Coat of pluralities of adhesive |
| | | | peptides/peptidomimetic |
| 36 | Multivalent adsorptive | 38 | Multivalent adsorptive |
| | radiocontrast nanoconstruct | | radiocontrast nanoconstruct |
| | (MARC) of triblock nano- | | (MARC) of triblock nano- |
| | spheres | | particles |
| 40 | Subcoat of linkers | 42 | Suitable catheter |

### REFERENCES

Agthoven, Johannes F Van, Jian-Ping Xiong, Jose Luis Alonso, Xianliang Rui, Brian D Adair, Simon L Goodman, and M Amin Arnaout. 2014. “Structural Basis for Pure Antagonism of Integrin α V β3 by a High-Affinity Form of Fibronectin.” Nature Structural & Molecular Biology 21 (4): 383-88.
Alon, Ronen, Edward A Bayer, and Meir Wilchek. 1990. “Streptavidin Contains an RYD Sequence Which Mimics the RGD Receptor Domain of Fibronectin.” Biochemical and Biophysical Research Communications 170 (3): 1236-41.
Bae, Kyongtae T. 2010. “Intravenous Contrast Medium Administration and Scan Timing at CT: Considerations and Approaches.” Radiology 256 (1): 32-61.

Boateng, Samuel Y, Syed S Lateef, William Mosley, Thomas J Hartman, Luke Hanley, and Brenda Russell. 2005. “RGD and YIGSR Synthetic Peptides Facilitate Cellular Adhesion Identical to That of Laminin and Fibronectin but Alter the Physiology of Neonatal Cardiac Myocytes.” American Journal of Physiology-Cell Physiology 288 (1): C30-38.
Calvete, Juan J. 1995. “On the Structure and Function of Platelet Integrin A11143, the Fibrinogen Receptor.” Proceedings of the Society for Experimental Biology and Medicine 208 (4): 346-60.
Hynes, Richard O. 2002. “Integrins: Bidirectional, Allosteric Signaling Machines.” Cell 110 (6): 673-87.
Ibanez, Borja, Stefan James, Stefan Agewall, Manuel J Antunes, Chiara Bucciarelli-Ducci, Hector Bueno, Alida L P Caforio, Filippo Crea, John A Goudevenos, and Sigrun Halvorsen. 2018. “2017 ESC Guidelines for the Management of Acute Myocardial Infarction in Patients Presenting with ST-Segment Elevation: The Task Force for the Management of Acute Myocardial Infarction in Patients Presenting with ST-Segment Elevation of the European Socie.” European Heart Journal 39 (2): 119-77.
Knight, C Graham, Laurence F Morton, Anthony R Peachey, Danny S Tuckwell, Richard W Farndale, and Michael J Barnes. 2000. “The Collagen-Binding A-Domains of Integrins A1β1 and A2β1recognize the Same Specific Amino Acid Sequence, GFOGER, in Native (Triple-Helical) Collagens.” Journal of Biological Chemistry 275 (1): 35-40.
Li, Feiya, Sambra D Redick, Harold P Erickson, and Vincent T Moy. 2003. “Force Measurements of the A5β1 Integrin—Fibronectin Interaction.” Biophysical Journal 84 (2): 1252-62.

McLane, Mary A, Elda E Sanchez, Alice Wong, Carrie Paquette-Straub, and John C Perez. 2004. “Disintegrins.” Current Drug Targets-Cardiovascular & Hematological Disorders 4 (4): 327-55.
Mould, A Paul, Stephanie J Barton, Janet A Askari, Susan E Craig, and Martin J Humphries. 2003. “Role of ADMIDAS Cation-Binding Site in Ligand Recognition by Integrin A5β1.” Journal of Biological Chemistry 278 (51): 51622-29.
Ruoslahti, Erkki. 1988. “Fibronectin and Its Receptors.” Annual Review of Biochemistry 57 (1): 375-413.
Sorenson, James A, and Michael E Phelps. 1987. Physics in Nuclear Medicine. Grune & Stratton New York.

Xiong, Jian-Ping, Thilo Stehle, Rongguang Zhang, Andrzej Joachimiak, Matthias Frech, Simon L Goodman, and M Amin Arnaout. 2002. “Crystal Structure of the Extracellular Segment of Integrin AVβ3 in Complex with an Arg-Gly-Asp Ligand.” Science 296 (5565): 151-55.
Zhu, Yunxiao, Zdravka Cankova, Marta Iwanaszko, Sheridan Lichtor, Milan Mrksich, and Guillermo A Ameer. 2018. “Potent Laminin-Inspired Antioxidant Regenerative Dressing Accelerates Wound Healing in Diabetes.” Proceedings of the National Academy of Sciences 115 (26): 6816-21.

### CONCLUSION

The proposed ARCs/MARCs are designed to have dual therapeutic and diagnostic actions which enhance the efficacy of real-time arterial dyeing during procedural angiography with or without percutaneous interventions. While the above description contains many specificities, these should not be construed as limitations on the scope of the invention, but rather as an exemplification of preferred embodiments thereof. Many other variations are possible. Accordingly, the scope of the invention should be determined not by the embodiments illustrated, but by the appended claims and their legal equivalents.

I claim in:

1. An adsorptive radiocontrast construct with dual therapeutic and diagnostic actions, comprising:
a radiodense nucleus, and
one or more adhesive arms tethered to said radiodense nucleus with or without a linker.

2. The adsorptive radiocontrast construct according to claim 1, wherein the radiodense nucleus comprises an element or molecule that is capable of absorbing or altering external electromagnetic radiation resulting in decreased exposure on an electromagnetic waves detector.

3. The adsorptive radiocontrast construct according to claim 1, wherein the adhesive arms comprise peptide motifs or peptidomimetics that bind to their corresponding receptors of cell-adhesion molecules.

4. The adsorptive radiocontrast construct according to claim 1, wherein the linker is a flexible molecule or a stretch of molecule of different lengths or polarity that can possess a branching capability, and can be used to link two or more molecules of interest together.

5. The adsorptive radiocontrast construct according to claim 1, whereby a concomitant therapeutic antiplatelet effect and a diagnostic real-time continuous visualization of an artery can be obtained via direct injection of the adsorptive radiocontrast constructs using a suitable catheter.

6. The adsorptive radiocontrast construct according to claim 1, wherein one or more additional therapeutically active molecules can be incorporated into its molecular structure, so that said additional therapeutically active molecules can be tethered to the radiodense nucleus, or anchored to or replace the linker.

7. The adsorptive radiocontrast construct according to claim 1, wherein the radiodense nucleus can be tethered to one adhesive arm resulting in a monovalent adsorptive radiocontrast construct, or can be tethered to multiple adhesive arms resulting in a multivalent adsorptive radiocontrast construct.

8. The adsorptive radiocontrast construct according to claim 1, wherein the adhesive arms can be identical resulting in a monospecific adsorptive radiocontrast construct, or different resulting in a polyspecific adsorptive radiocontrast construct.

9. The adsorptive radiocontrast construct according to claim 1, can be used for procedural real-time angiography with or without percutaneous intervention.

10. An adsorptive radiocontrast construct with dual therapeutic and diagnostic actions, comprising one or more adhesive peptides or peptidomimetics that contain within its molecular structure a radiodense element or molecule.

11. The adsorptive radiocontrast construct according to claim 10, wherein the adhesive peptides or peptidomimetics bind to their corresponding receptors of cell-adhesion molecules.

12. The adsorptive radiocontrast construct according to claim 10, wherein the radiodense element or molecule is capable of absorbing or altering external electromagnetic radiation resulting in decreased exposure on an electromagnetic waves detector.

13. The adsorptive radiocontrast construct according to claim 10, wherein the adhesive peptides or peptidomimetics, that contain within its molecular structure a radiodense element or molecule, can be singles resulting in a monovalent adsorptive radiocontrast construct, or can be multiple tethered to each other with flexible linkers resulting in a multivalent adsorptive radiocontrast construct.

14. The adsorptive radiocontrast construct according to claim 10, wherein the adhesive peptides or peptidomimetics, that contain within its molecular structure a radiodense element or molecule, can be identical resulting in a monospecific adsorptive radiocontrast construct, or different resulting in a polyspecific adsorptive radiocontrast construct.

15. A multivalent adsorptive radiocontrast nanoconstruct with dual therapeutic and diagnostic actions, comprising:
a radiodense nanocore, that is coated with
a coat of pluralities of adhesive peptides or peptidomimetics with or without a subcoat of linkers, wherein said pluralities of adhesive peptides or peptidomimetics are presented to interact simultaneously with multiple corresponding binding sites.

16. The multivalent adsorptive radiocontrast nanoconstruct according to claim 15, wherein the radiodense nanocore comprises an element or molecule that is capable of absorbing or altering external electromagnetic radiation resulting in decreased exposure on an electromagnetic waves detector.

17. The multivalent adsorptive radiocontrast nanoconstruct according to claim 15, wherein the pluralities of adhesive peptides or peptidomimetics bind to their corresponding receptors of cell-adhesion molecules.

18. The multivalent adsorptive radiocontrast nanoconstruct according to claim 15, wherein the subcoat of linkers can comprise a biodegradable layer of fluidic material to which the pluralities of adhesive peptides or peptidomimetics are tethered, whereby the pluralities of adhesive peptides or peptidomimetics retain unrestricted mobility, efficient clustering, and redistribution.

19. The multivalent adsorptive radiocontrast nanoconstruct according to claim 15, wherein the pluralities of adhesive peptides or peptidomimetics, can be identical resulting in a monospecific multivalent adsorptive radiocontrast nanoconstruct, or different resulting in a polyspecific multivalent adsorptive radiocontrast nanoconstruct.

20. The multivalent adsorptive radiocontrast nanoconstruct according to claim 15, can be a nanosphere or a nanoparticle with anisometric dimensions.
